(19) United States
(12) Patent Application Publication
(10) Pub. No.: US 2024/0036039 A1
(43) Pub. Date: Feb. 1, 2024

(54) SHELF-STABLE, READY-TO-USE, ELECTROCHEMICAL APTAMER SENSORS

(71) Applicant: University of Cincinnati, Cincinnati, OH (US)

(72) Inventors: Jason Heikenfeld, Cincinnati, OH (US); Mark Friedel, Cincinnati, OH (US); Zachary Watkins, Cincinnati, OH (US)

(21) Appl. No.: 18/028,030

(22) PCT Filed: Oct. 2, 2020
(86) PCT No.: PCT/US2020/054041

§ 371 Date: Mar. 23, 2023

Related U.S. Application Data
(60) Provisional application No. 62/911,043, filed on Oct. 4, 2019.

(51) Int. Cl. G01N 33/543; C12N 15/115

(52) U.S. Cl. CPC G01N 33/5438; C12N 15/115; C12N 2310/16

(57) ABSTRACT

Described are sensing devices and methods for detecting at least one analyte in a sample solution. The sensor is capable of a pre-storage state, a storage state and a sensing state. The sensor includes at least one substrate-adjacent aptamer; where the sensor, in the absence of a material to reduce or prevent degradation, would incur significant degradation when the sensor is placed in a storage state. The sensor further includes at least one material which reduces or prevents the significant degradation during the storage state.

### CROSS REFERENCE TO RELATED APPLICATIONS

This application claims priority to U.S. Provisional Application Ser. No. 62/911,043, filed Oct. 4, 2019, which application is hereby incorporated by reference in its entirety.

### TECHNICAL FIELD

The present invention relates to electrochemical aptamer sensors. More specifically, this invention relates to shelf-stable, ready to use electrochemical aptamer sensors.

### BACKGROUND OF THE INVENTION

Biosensors based on aptamers as biorecognition elements are useful as a diagnostic tool. Selected aptamers bind their targets with affinities and specificities that can be comparable to those of antibodies. Aptamers present some advantages compared to antibodies, especially accurate and reproducible quantitative detection, especially over multiple measurements. Moreover, aptamers offer chemical stability under a wide range of buffer conditions, are resistant to harsh treatments without losing their bioactivity, and thermal denaturation is reversible for aptamers. Electrochemical devices have received considerable recent attention in connection to the transduction of aptamer interactions. Electrochemical transduction presents considerable advantages over optical, piezoelectric or thermal detection. Electrochemical detection offers a variety of advantages, including high sensitivity and selectivity, compatibility with novel microfabrication technologies, inherent miniaturization, low cost, disposability, simple-to-operate, robust, low power requirements, and independence of sample turbidity. Despite these advantages, electrochemical sensors using substrate-adjacent aptamers can degrade during storage. When electrochemical sensors using substrate-adjacent aptamers are placed in a sample solution to be tested, they often undergo initial signal changes as the test fluid alters the sensor itself in both negative ways (aptamer damage) and positive ways (fouling that can reduce background current). Therefore, a need still exists for a means to improve the stability of electrochemical aptamer sensors during storage and store them in a manner such that they are ready for use without any preconditioning.

### SUMMARY OF THE INVENTION

The present invention addresses this need by including at least one additional material to an electrochemical aptamer sensor, reducing or preventing this degradation, and or reducing or preventing the need for preconditioning of the sensor prior to use. In one embodiment, the present invention is a sensor for at least one analyte in a sample solution. The sensor is capable of a pre-storage state, a storage state and a sensing state. Further, the sensor includes at least one substrate-adjacent aptamer. In addition, the sensor includes at least one storage material, which reduces or prevents significant degradation during the storage state such that the sensor can then be used in the sensing state after the storage state. Also, in the absence of a material to reduce or prevent degradation, the sensor would incur significant degradation when the sensor is placed in a storage state. Further, the sensor is capable of being in a storage state for at least one month.

In one embodiment, after the sensor has been in a sensing state, it has an initial electrochemical signal gain that is within at least one of 30 percent of the signal gain in the pre-storage state. In another embodiment, after the sensor has been in a sensing state, it has an initial electrochemical signal gain that is within 20 percent of the signal gain in the pre-storage state. In one embodiment, after the sensor has been in a sensing state, it has an initial electrochemical signal gain that is within 10 percent of the signal gain in the pre-storage state. In another embodiment, after the sensor has been in a sensing state, it has an initial electrochemical signal gain that is within 5 percent of the signal gain in the pre-storage state.

In one embodiment, the sensor is capable of being in a storage state at least 6 months. In another embodiment, the sensor is an electrochemical aptamer sensor with an attached redox couple, and further, wherein the substrate is an electrode. In one embodiment, the sensor also includes a plurality of substrate-adjacent molecules that passivate the electrode surface. In another embodiment, the storage material prevents significant degradation of the substrate-adjacent molecules.

In one embodiment, the storage material is a solid material in the storage state that is dissolvable during or prior to the sensing state. In another embodiment, the storage material comprises a sugar.

In one embodiment, the storage material comprises one or more polymers. In another embodiment, the storage material comprises a biomolecule or a denatured biomolecule. In one embodiment, the storage material comprises a non-aqueous fluid. In another embodiment, the storage material comprises an inert gas. In one embodiment, the storage material comprises vacuum.

In another embodiment, the sensor further includes at least one recovery material. In one embodiment, the recovery material is a fluid that dissolves the storage material. In another embodiment, the sensor further includes at least one pre-conditioning material. In one embodiment, the pre-conditioning material exists in the pre-storage state. In another embodiment, the pre-conditioning material exists in the storage state. In one embodiment, the pre-conditioning material exists in both the pre-storage and the storage state. In another embodiment, the pre-conditioning material comprises at least one biomolecule. In one embodiment, the pre-conditioning material consists essentially of solutes found in serum. In another embodiment, the pre-conditioning material consists essentially of solutes found in denatured serum.

In one embodiment, after the sensor has been in a storage state, it has an initial electrochemical signal gain that when measured within the first 5 minutes is within 30 percent of a steady state electrochemical response for the sensor. In another embodiment, after the sensor has been in a storage state, it has an initial electrochemical signal gain that when measured within the first 5 minutes is within 20 percent of a steady state electrochemical response for the sensor. In one embodiment, after the sensor has been in a storage state, it has an initial electrochemical signal gain that when measured within the first 5 minutes is within 10 percent of a steady state electrochemical response for the sensor. In another embodiment, after the sensor has been in a storage state, it has an initial electrochemical signal gain that when measured within the first 5 minutes is within 5 percent of a steady state electrochemical response for the sensor. In one embodiment, after the sensor has been in a storage state, it has an initial electrochemical signal gain that when measured within the first 15 minutes is within 30 percent of a steady state electrochemical response for the sensor. In another embodiment, after the sensor has been in a storage state, it has an initial electrochemical signal gain that when measured within the first 15 minutes is within 20 percent of a steady state electrochemical response for the sensor. In one embodiment, after the sensor has been in a storage state, it has an initial electrochemical signal gain that when measured within the first 15 minutes is within 10 percent of a steady state electrochemical response for the sensor. In another embodiment, after the sensor has been in a storage state, it has an initial electrochemical signal gain that when measured within the first 15 minutes is within 5 percent of a steady state electrochemical response for the sensor.

In another embodiment of the present invention, a method of detecting at least one analyte in a sample solution is disclosed. The method involves obtaining a sensor in a pre-storage state, wherein the sensor comprises at least one substrate-adjacent aptamer and at least one first material which reduces or prevents significant degradation during a storage state. Then, the sensor is stored in a storage state, then placed in a sensing state. The sample solution is then exposed to the sensor. Additionally, the sensor, in the absence of a material to reduce or prevent degradation, would incur significant degradation when the sensor is placed in a storage state.

In one embodiment, after the sensor has been in a sensing state, it has an initial electrochemical signal gain that is within at least one of 30 percent of the signal gain in the pre-storage state. In another embodiment, after the sensor has been in a sensing state, it has an initial electrochemical signal gain that is within 20 percent of the signal gain in the pre-storage state. In one embodiment, after the sensor has been in a sensing state, it has an initial electrochemical signal gain that is within 10 percent of the signal gain in the pre-storage state. In another embodiment, after the sensor has been in a sensing state, it has an initial electrochemical signal gain that is within 5 percent of the signal gain in the pre-storage state.

In one embodiment, vacuum is applied before or during the storage state. In another embodiment, a recovery material is applied after the storage state to remove the storage material. In one embodiment, a pre-conditioning material is applied during at least the pre-storage or storage states.

In another embodiment, after the sensor has been in a storage state, it has an initial electrochemical signal gain that when measured within the first 15 minutes is within 30 percent of a steady state electrochemical response for the sensor. In one embodiment, after the sensor has been in a storage state, it has an initial electrochemical signal gain that when measured within the first 15 minutes is within 20 percent of a steady state electrochemical response for the sensor. In another embodiment, after the sensor has been in a storage state, it has an initial electrochemical signal gain that when measured within the first 15 minutes is within 10 percent of a steady state electrochemical response for the sensor. In one embodiment, after the sensor has been in a storage state, it has an initial electrochemical signal gain that when measured within the first 15 minutes is within 5 percent of a steady state electrochemical response for the sensor. In another embodiment, the pre-conditioning material comprises at least one biomolecule. In one embodiment, the pre-conditioning material comprises primarily the solutes found in serum.

These and other embodiments of the disclosed invention are directed to materials and methods that create shelf-stable aptamer sensors.

### BRIEF DESCRIPTION OF THE DRAWINGS

The objects and advantages of the disclosed invention will be further appreciated in light of the following detailed descriptions and drawings in which:

FIG. 1A is a signal-gain plot and FIG. 1B is a voltammogram of the redox-couple peak for an aptamer sensor without benefit of the present invention.

FIG. 2A is a signal-gain plot and FIG. 2B is a voltammogram of the redox-couple peak for an aptamer sensor with benefit of a storage state of the present invention.

FIG. 3A is a signal-gain plot and FIG. 3B is a voltammogram of the redox-couple peak for an aptamer sensor with benefit of a storage state of the present invention.

FIG. 4A is a signal-gain plot and FIG. 4B is a voltammogram of the redox-couple peak for an aptamer sensor with benefit of a storage state of the present invention.

FIG. 5 is a normalized signal gain plot for an aptamer sensor with the benefit of a storage state of the present invention but without the benefit of preconditioning of the present invention.

FIG. 6 is a normalized signal gain plot for an aptamer sensor with the benefit of a storage state of the present invention and with benefit of preconditioning of the present invention.

FIG. 7 is a normalized signal gain plot for an aptamer sensor with the benefit of a storage state of the present invention and with benefit of preconditioning of the present invention.

### DEFINITIONS

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

As used herein, the term “aptamer” means a molecule that undergoes a conformation change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function.

As used herein, “electrochemical aptamer sensor” means a sensor that provides at least one or a plurality of measurements over time of a property of a sample fluid, and which comprises a plurality of electrode-bound aptamers for signal transduction. Such aptamer sensors can rely on an attached redox couple or other signal transduction mechanisms.

As used herein, “substrate-adjacent aptamer” refers to a plurality of aptamers that are chemically bonded to, or retained in direct proximity to, a substrate and/or a material on a substrate such as an electrode. “Bound” refers to the aptamers remaining adjacent to the substrate when placed in a sample solution. A substrate-bound aptamer may further include at least an electrochemical reporter that is attached to the aptamer, such as a redox couple, or a fluorescent tag.

As used herein, “substrate-adjacent molecule” refers to a plurality of molecules that are chemically bonded to, or retained in direct proximity to, a substrate and/or a material on a substrate such as an electrode. A substrate-adjacent molecule serves a different function than a substrate-adjacent aptamer. For example, passivation of aptamer-containing gold surfaces with a substrate-adjacent molecule such as mercapto hexanol (MCH) has been reported to decrease electrical background current and decrease non-specific binding presumably by filling in the gold regions left exposed after aptamer assembly.

As used herein, “storage material” refers to at least one chemical, fluid, material, or combination thereof, which stabilizes substrate-bound aptamers, and/or substrate-adjacent molecules, such that the sensor can be stored without significant degradation prior to use, and the storage material removed prior to use of the sensor by means such as evaporation, dissolution, or other suitable mechanism. Non-limiting examples of such storage materials include materials that are have low concentrations of or are devoid of water, oxidizing agents, acids, bases, and storage materials may be chemicals, fluids, gases, sugars, biofilms, or materials or combinations of materials that can degrade a sensor. Optionally, storage materials may also include materials that are have low concentrations of or are devoid of oxygen or other reactive materials. A storage material could be vacuum.

As used herein, “sample fluid” or “sample solution” refers to any liquid or fluid which contains at least one analyte that is to be measured in presence, change, concentration, or other measurement, by a sensor specific to that analyte. In one embodiment, a sample or sample solution is a biofluid. In other embodiments, a sample or sample solution is water from the environment, manufacturing fluid for food, or other types of sample solutions that would benefit from the disclosed invention. Non-limiting examples of sample solutions include river water, food processing fluids, human blood, or other solutions. As used herein, “biofluid” means a fluid source of sample solution with analytes originating in the human body. For example, sweat is a biofluid source of analytes that is from eccrine or apocrine glands. In another embodiment, a biofluid is a solution that bathes and surrounds tissue cells such as interstitial fluid. Non-limiting examples of biofluid include blood, interstitial fluid, saliva, tears, or other possible biofluids.

As used herein, “preconditioning or preconditioning material” refers to at least one chemical, fluid, material, or combination thereof, which preconditions the aptamer sensor such that typical initial changes in electrochemical signal for the aptamer sensor when it is placed in sample fluid are mitigated such the aptamer sensor is immediately or quickly ready to use without need of waiting for preconditioning.

As used herein, “steady state electrochemical response or signal” is the sensor signal that exists after preconditioning of the sensor.

As used herein, “storage state” refers to placing the sensor in a material or absence of material during storage, and therefore in the absence of sample solution. For example, storage states may include and are not limited to vacuum, aqueous fluids, non-aqueous fluids, nitrogen, sugars, biofilms, argon, air, or other suitable storage material.

As used herein, “pre-storage state” refers to a state of the sensor at a time before the storage state.

As used herein, “pre-conditioning material” refers to a material that in the pre-storage or storage states pre-conditions the sensor such that it is ready to use in the sensing state.

As used herein, “sensing state” refers to a state of the sensor after the time of storage and during use for sensing an analyte in a sensor solution. In addition, the “sensing state” refers to normal operation of the sensor once it is ready for accurate measurement of at least one analyte.

As used herein, “recovery material” refers to a material that a sensor is placed in after the storage state to prepare the sensor for the sensing state. In one embodiment, a recovery material is a non-aqueous solvent that dissolves the storage material, or which recovers the substrate-adjacent aptamers such that they operate properly in the sensing state. In another embodiment, the recovery material is a slightly alkaline buffer such as TE buffer: Tris, a common pH buffer, and EDTA, a molecule that chelates cations like Mg2+. TE buffer is able to solubilize DNA or RNA, while protecting it from degradation. In another embodiment, the recovery material is denatured serum.

### DETAILED DESCRIPTION OF THE INVENTION

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention, but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

Embodiments of the disclosed invention are directed to aptamer sensors that are shelf-stable and ready to use without preconditioning. Certain embodiments of the disclosed invention show sensors as simple individual elements. It is understood that many sensors require two or more electrodes such as working and counter electrodes, reference electrodes, or additional supporting technology or features which are not captured in the description herein. Sensors measure a characteristic of an analyte. Sensors are preferably electrical in nature, but may also include optical, chemical, mechanical, or other known biosensing mechanisms. Sensors can be sensors such as electrochemical aptamer sensors that sense analytes such as cortisol, vasopressin, or IL-6, drugs, or other analytes for example. Sensors can be in duplicate, triplicate, or more, to provide improved data and readings. Sensors may provide continuous or discrete data and/or readings. Certain embodiments of the disclosed invention may show or refer to sub-components of what would be sensing devices with more sub-components needed for use of the device in various applications, which are known (e.g., a battery, antenna, adhesive, electronics, etc.), and for purposes of brevity and focus on inventive aspects, such components may not be explicitly shown in the diagrams or described in the embodiments of the disclosed invention.

With reference to an embodiment of the disclosed invention, aptamer sensors based on substrate-adjacent aptamers can readily degrade during storage. Significant degradation modes include but are not limited to losing bonding to the substrate, binding of adjacent molecules on the substrate, and chemical or physical changes in the aptamers, molecules, or chemicals on the substrate. A common strategy for making aptamer sensors in the pre-storage state is to functionalize a gold electrode with an aptamer by thiol bonding to the gold, with the aptamer having a redox couple which acts as an electrochemical reporter. Such sensors also often employ a blocking layer, or additional molecules, that improve performance and function of the sensor when placed in a sample solution. All of these molecules found on the substrate can be subject to significant degradation during a storage state. In addition, all of these molecules found on the substrate can be modified, or the sensor itself initially modified, as the sensor is first placed into sample fluid.

In some embodiments the present invention includes at least one material in the pre-storage, storage, or sensing states which prevents significant degradation during the storage state, for a sensor which would incur significant degradation during the storage state without such an additional material, and/or the material preconditions the device such that it is ready to use.

With further reference to an embodiment of the disclosed invention, the storage state is, in alternative embodiments, at least 1 week, at least 1 month, at least 6 months, or at least 1 year.

With further reference to an embodiment of the disclosed invention, the substrate-adjacent aptamer is an electrochemical aptamer that is bound to an electrode and the aptamer has an attached redox couple. A non-limiting example of a redox couple is methylene blue. In one embodiment, the substrate-adjacent aptamer is an optical aptamer with a fluorescent tag and fluorescent quencher that is held adjacent to a substrate by a membrane such as dialysis membrane which is permeable to an analyte such as cortisol but which is impermeable to the much larger aptamer molecule.

In another embodiment of the disclosed invention, the sensor further includes at least one substrate-adjacent molecule. In one embodiment, the substrate-adjacent molecule is mercapto hexanol, for passivation of aptamer-containing gold surfaces to decrease non-specific binding presumably by filling in the gold regions left exposed after aptamer assembly. If the substrate-adjacent molecule is significantly degraded in the storage state, then at least one additional first material can be added to prevent significant degradation.

In one embodiment, a first material is applied, as for example a sucrose solution is applied during the pre-storage state, then vacuum dried, then the sensor stored in vacuum or nitrogen, an environment without substantial or any reactive species, such that the substrate-adjacent aptamer and substrate-adjacent molecule are preserved in their structure and separated from adjacent molecules by the first material sucrose. In this manner, the sucrose replaces the function of water that normally provides a separating material between adjacent molecules and adjacent aptamers. Sucrose may also hold the aptamer or other molecules in place, such that if during storage they lose bonding with the substrate they are on, they can rebond with it during storage. The addition of a first material may prevent degradation for the substrate adjacent aptamer, the substrate adjacent molecule, or other materials or features of the sensor. In another embodiment, the first material is a molecule, with sucrose being an example of a molecule. In another embodiment, the first material is a molecule that is substrate adjacent and potentially bound to the substrate, with molecule choice and/or density of the molecule preventing significant degradation.

In one embodiment of the disclosed invention, the first material is a storage material in the storage state. Non-limiting examples of storage materials include sucrose, nitrogen, TE buffer, antimicrobial agents, or other suitable storage materials. In one embodiment, the storage material is a fluid or a liquid, such as glycerin, or glycols. In another embodiment, the storage material is a solid such as sucrose or polyvinylalchohol that is applied in solution or liquid form in the pre-storage state, then made solid for the storage state, then removed by dissolution in the sensing-state. The dissolution can be in the sample solution or in another solution such as recovery material, for example a polymer that is dissolvable in ethanol or ethanol-water mix then the ethanol and dissolved polymer are removed by an aqueous sample solution. The storage material may also be a chemical or molecule.

In an embodiment of the disclosed invention, the storage material is in total or in part an inert gas such as argon, nitrogen, carbon-dioxide, or other non-reactive gas or a gas that is antimicrobial in nature.

With further reference to an embodiment of the disclosed invention, flash-freezing is a process in which the sensor is quickly cooled to below freezing temperatures (<−20 degree Celsius). After the rapid freezing, the sensor can be stored in a standard −20 C freezer. Therefore, one embodiment of the present invention includes a storage material that is comprised at least in part of water or ice. In another embodiment, the present invention includes a storage material that is comprised at least in part of a solvent such as glycols or other solvents.

With further reference to an embodiment of the disclosed invention, wherein after the sensor has been in a sensing state, it has an initial electrochemical signal gain that is within at least one of 30, 20, 10, or 5 percent of the signal gain in the pre-storage state.

Further, in some embodiments of the present invention, regardless of the storage state, in some cases a steady state electrochemical response may not be immediate. This is due to the materials in the sensor itself and/or the storage state materials not readily reconstituting in sample fluid or recovering in sample fluid. Therefore, a recovery material may be used. For example, in one embodiment the storage material is a polymer that has higher solubility at pH=8. In this embodiment, the recovery material is a buffer solution of pH=8 and it is used initially before the sensing state. In another embodiment, the recovery material is a material such as alkaline buffer. Non-limiting examples of alkaline buffers include TE buffer Tris, a common pH buffer, and EDTA, a molecule that chelates cations like Mg2+. TE buffer is able to solubilize DNA or RNA, while protecting it from degradation. In one embodiment, the recovery material is added to the sample fluid. As a non-limiting example, the sample fluid is serum and the recovery material is MgCl salt that is dissolved in the serum to improve sensor response but also to cause a more rapid recovery of the device such that it reaches steady state signal after storage. In another embodiment, the recovery material is a solvent such as decanol, which serves to recover storage induced degradation to the substrate-adjacent molecule such as mercapto hexanol. Therefore, the recovery material may apply to the recovery of substrate adjacent aptamers and/or substrate adjacent molecules.

The following examples are provided to help illustrate the disclosed invention, and are not comprehensive or limiting in any manner. These examples serve to illustrate that although the specification herein does not list all possible device features or arrangements or methods for all possible applications, the invention is broad and may incorporate other useful methods or aspects of materials, devices, or other embodiments for the broad applications of the disclosed invention.

### EXAMPLES

### Example 1

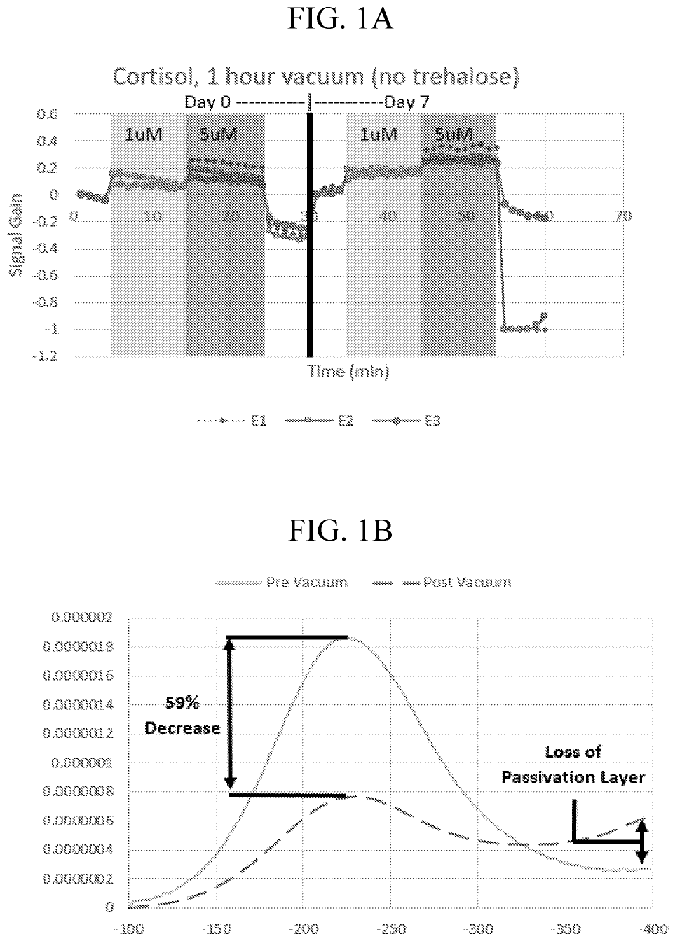

With reference to FIGS. 1A and 1i, an aptamer sensor for cortisol was tested where the aptamer sensor was fully fabricated (three electrodes E1, E2, E3 were tested). It was tested (day 0) in a sample fluid of buffer solution with or without cortisol, dried in vacuum for 1 hour, and stored in a container with air for 1 week prior to retesting. As can be seen, the signal is both more noisy (FIG. 1A) and the signal peak weaker (FIG. 1B) and there is evidence of loss or damage to the mercaptohexanol passivating layer by increased background current (FIG. 1B).

### Example 2

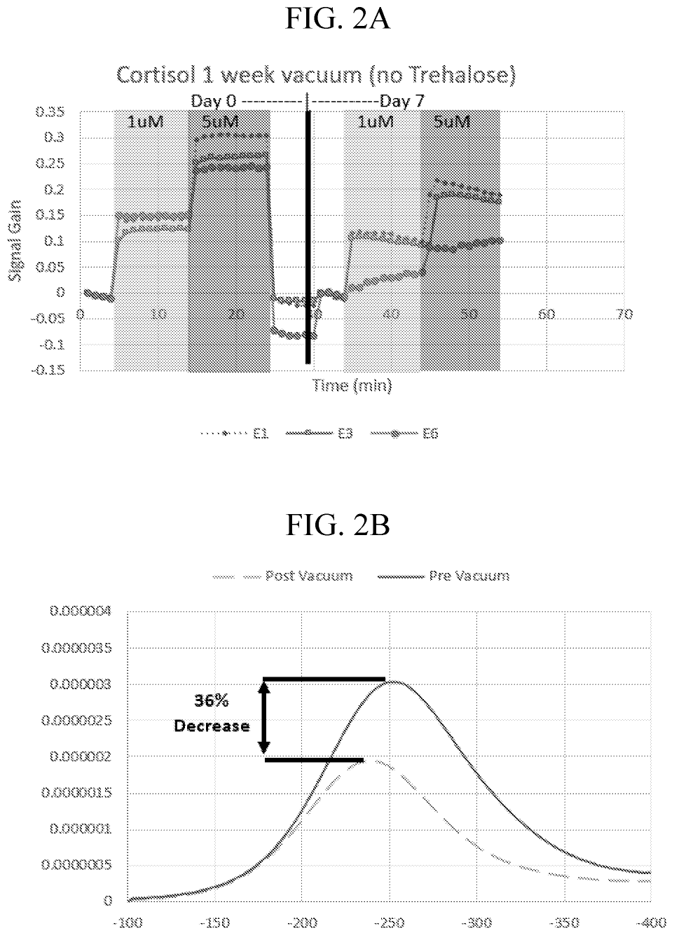

With reference to FIGS. 2A and 2B, an identical device was fabricated and tested initially in a pre-storage state, and when vacuumed to dry the device, the device remained in vacuum of less than 1 Torr for 1 week as a storage state. In this example, the storage state is a state void of any material except material comprising the aptamer sensor itself (e.g. vacuum), where vacuum is the storage material. During this vacuum storage the degradation of the device is significantly reduced during the retesting one week later which is referred to as the sensing state. Various aptamer sensors and aptamers can give varying results of storage stability, and therefore may be stored for at least one of 1 week, 1 month, 6 months, 1 year, and provide a reduction in signal gain that is less than at least one of 2%, 5%, 10%, 20%, 30%, 40%, or 80% of the signal gain before storage.

### Example 3

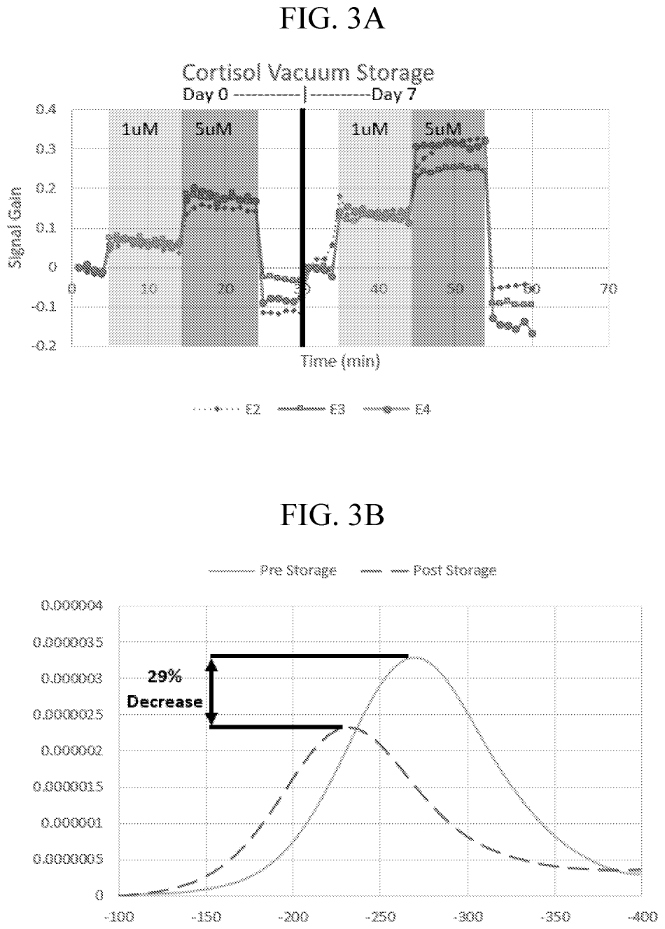

With reference to FIGS. 3A and 3B, an identical device was fabricated and initially tested, the device was then coated with trehalose as a storage material (which is an example of a sugar or starch coating) and stored in vacuum for 1 week, again resulting in reduced damage to the device once it was retested. In this example an embodiment with at least one storage material is shown. In this embodiment, the storage material is a sugar, and specifically trehalose.

### Example 4

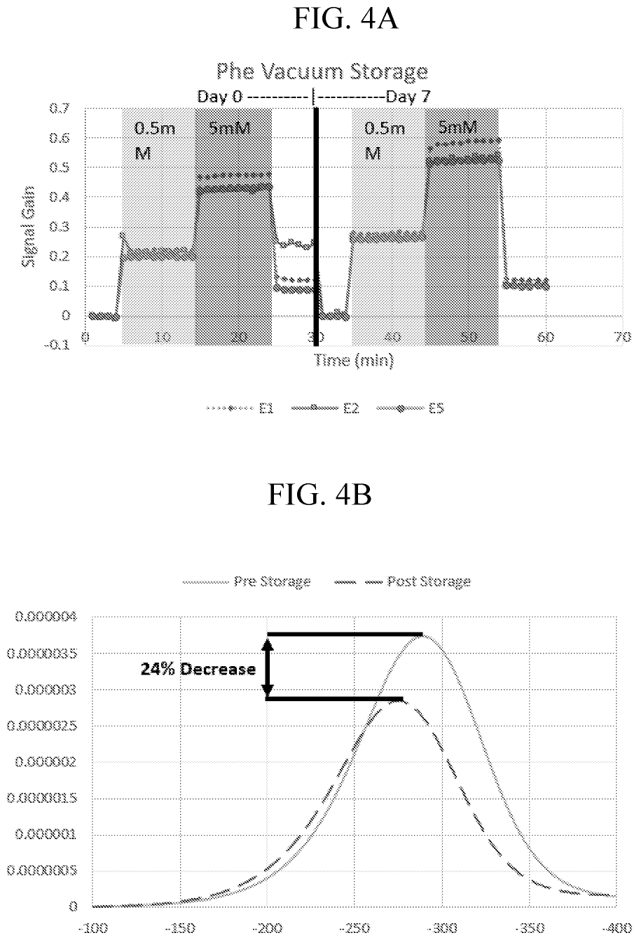

With reference to FIGS. 4A and 4B, an identical experiment to that of FIGS. 3A and 3B was performed with the exception that an aptamer for phenylalanine was used. This experiment shows that present invention applies broadly to aptamer sensors.

### Example 5

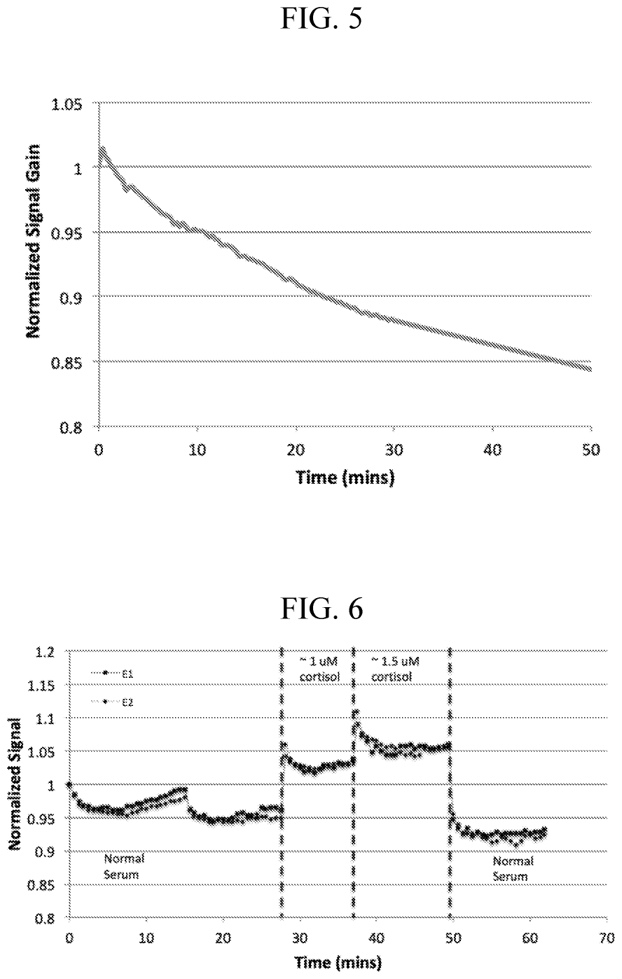

With reference to FIG. 5, a device identical to that of FIGS. 3A and 3B was tested, and the initial signal gain vs. time is shown where the sample fluid is serum, a biofluid. Serum contains numerous solutes that can bind to, foul, stabilize, and otherwise alter the electrochemical response of an aptamer sensor. All aptamer sensors have some signal drift and some signal degradation over time such as hours, but this initial signal change over minutes to 10's of minutes is different as the sensor begins the sensing state. Conventional sensors can therefore require preconditioning in the target sample fluid before a useful sensor result can be acquired. This can confound efforts to calibrate sensors and can make them simply not ready-to-use.

### Example 6

With reference to FIG. 6, an identical device to FIG. 5 was tested, and underwent preconditioning using a pre-conditioning material of serum for 150 minutes in the pre-storage state. The sensor was then made shelf stable with trehalose as a storage material and tested in the sensing state. It had a reduced initial change in normalized signal gain and therefore was more immediately ready for use. Serum can contain proteases or other solutes that can damage an aptamer sensor, and therefore, in one embodiment, denatured serum is used. One of the benefits of serum is stabilization of the passivating layer through solutes such as albumin, which reduce electrochemical background current. Therefore the pre-conditioning material may be the sample fluid itself, or may be a fluid with at least one solute that preconditions the device, such that when tested, the device satisfies either one of: stabilizing to a normal steady state electrochemical signal gain in at least one of less than 30 minutes, 10 minutes, 5 minutes, 2 minutes, or 1 minute; having an initial electrochemical signal gain that when measured within the first 5 minutes or 15 minutes is at least one of 50%, 20%, 10%, 5%, or 2% within the steady state electrochemical response.

### Example 7

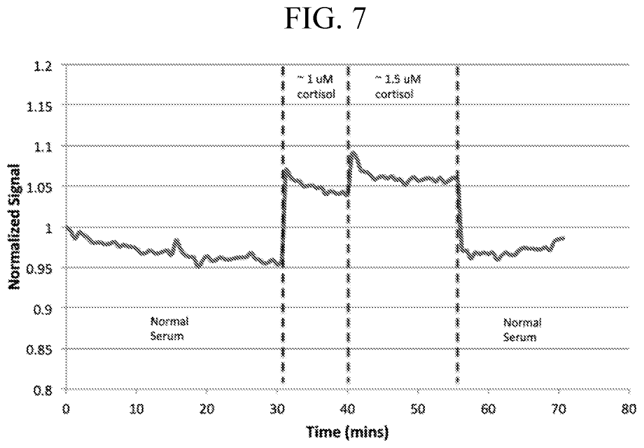

With reference to FIG. 7, and with reference to FIG. 6 where the storage material was distinct from the preconditioning material and/or the sample fluid, a result is shown where the storage material, sample fluid, and/or the preconditioning material are similar in at least one property that provides an initial electrochemical signal gain that when measured within the first 5 minutes or 15 minutes is at least one of 50%, 20%, 10%, 5%, or 2% within the steady state electrochemical response. In this case, the precondition material was serum, the device was vacuum dried with serum on it as the storage material, and the sample fluid was serum. As a result, when the device is placed in the sensing state, dissolving away a material not prevalent in the sensing state such as trehalose is not required and the device is even more ready to use as a result. In one embodiment, where all three states have serum as described above, they are similar in just one material or solute. In another embodiment, they are similar in multiple materials or solutes, such as albumin, and/or an amino acid, etc.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

1-51. (canceled)

52. A sensor for at least one analyte in a sample solution, said sensor being capable of a pre-storage state, a storage state and a sensing state; the sensor comprising;
a. at least one substrate-adjacent aptamer; and
b. at least one storage material which reduces or prevents significant degradation during the storage state such that the sensor can then be used in the sensing state after the storage state;
wherein the sensor, in the absence of a material to reduce or prevent degradation, would incur significant degradation when the sensor is placed in a storage state, and further, wherein the sensor is capable of being in a storage state for at least one month.

53. The sensor of claim 52, wherein after the sensor has been in a sensing state, it has an initial electrochemical signal gain that is within at least one of 30 percent of the signal gain in the pre-storage state.

54. The sensor of claim 52, wherein after the sensor has been in a sensing state, it has an initial electrochemical signal gain that is within 20 percent of the signal gain in the pre-storage state.

55. The sensor of claim 52, wherein the sensor is capable of being in a storage state at least 6 months.

56. The sensor of claim 55, wherein after the sensor has been in a sensing state, it has an initial electrochemical signal gain that is within at least one of 30 percent of the signal gain in the pre-storage state.

57. The sensor of claim 55, wherein after the sensor has been in a sensing state, it has an initial electrochemical signal gain that is within 20 percent of the signal gain in the pre-storage state.

58. The sensor of claim 52, wherein the sensor is an electrochemical aptamer sensor with an attached redox couple, and further, wherein the substrate is an electrode.

59. The sensor of claim 58, further comprising a plurality of substrate-adjacent molecules that passivate the electrode surface.

60. The sensor of claim 59, wherein the storage material prevents significant degradation of the substrate-adjacent molecules.

61. The sensor of claim 52, wherein the storage material is a solid material in the storage state that is dissolvable during or prior to the sensing state.

62. The sensor of claim 61, wherein the storage material comprises a sugar.

63. The sensor of claim 61, wherein the storage material comprises one or more polymers.

64. The sensor of claim 61, wherein the storage material comprises a biomolecule or a denatured biomolecule.

65. The sensor of claim 52, further comprising at least one recovery material.

66. The sensor of claim 65, wherein the recovery material is a fluid that dissolves the storage material.

67. The sensor of claim 52, further comprising at least one pre-conditioning material.

68. The sensor of claim 67, wherein the pre-conditioning material exists in the pre-storage state.

69. The sensor of claim 67, wherein the pre-conditioning material exists in the storage state.

70. The sensor of claim 67, wherein after the sensor has been in a storage state, it has an initial electrochemical signal gain that when measured within the first 5 minutes is within 30 percent of a steady state electrochemical response for the sensor.

71. The sensor of claim 67, wherein after the sensor has been in a storage state, it has an initial electrochemical signal gain that when measured within the first 5 minutes is within 20 percent of a steady state electrochemical response for the sensor.
